(19) United States
(12) Patent Application Publication
(10) Pub. No.: US 2024/0327367 A1
(43) Pub. Date: Oct. 3, 2024

(54) PROCESS FOR PREPARING A OXACYLOHEXANE OR OXACYLOPENTANE DERIVATIVE

(71) Applicant: Firmenich SA, Satigny (CH)

(72) Inventors: Pierre-Yves DAPSENS, Satigny (CH); Lea VILCOQ, Satigny (CH)

(21) Appl. No.: 18/293,768

(22) PCT Filed: Jul. 28, 2022
(86) PCT No.: PCT/EP2022/071263

§ 371 Date: Jan. 30, 2024

(30) Foreign Application Priority Data
Jul. 30, 2021 (EP) 21188887.0

(51) Int. Cl. C07D 307/92; C11B 9/00

(52) U.S. Cl. CPC C07D 307/92; C11B 9/0076

(57) ABSTRACT

The present invention relates to the field of organic synthesis and, more specifically, it concerns a process for the preparation of a cycloether of formula (I) comprising the cyclisation of compound of formula (II) performed in the presence of heterogenous acidic catalyst.

### TECHNICAL FIELD

The present invention relates to the field of organic synthesis and, more specifically, it concerns a process for the preparation of a cycloether of formula (I) comprising the cyclisation of compound of formula (II) performed in the presence of heterogenous acidic catalyst.

### BACKGROUND

The cycloether derivatives represent skeletons highly desirables which could be used as such or as key intermediates useful to prepare more complex compounds in different fields such as, among others, perfumery, cosmetic, pharmaceutic or agrochemistry. Relevant cycloether derivatives in perfumery industry are, for example, Cetalox® (3a,6,6,9a-tetramethyldodecahydronaphtho[2,1-b]furan; origin: Firmenich SA, Geneva, Switzerland) or Ambrox ((3aR,5aS,9aS,9bR)-3a,6,6,9a-tetramethyldodecahydronaphtho[2,1-b]furan; origin: Firmenich SA, Geneva, Switzerland) being a key constituent of natural ambergris. Those perfuming ingredients represent some of the most sought-after ingredients in the perfumery industry. Several alternative processes to prepare Cetalox® or Ambrox® have been developed and in particular via a cyclisation reaction of 2-(5,5,8a-trimethyl-2-methylenedecahydronaphthalen-1-yl)ethan-1-ol as a last step. The cyclisation has been disclosed under acidic conditions providing low yield or low selectivity due to the isomerisation of the double bond of the starting material or the formation of the undesired diastereoisomer(s).

In the meantime, today there is a need to foster sustainable processes, for examples using recyclable catalysts such as heterogenous catalysts. The cyclisation reaction of 2-(5,5,8a-trimethyl-2-methylenedecahydronaphthalen-1-yl)ethan-1-ol in the presence of heterogeneous catalysts, to produce Cetalox® or Ambrox®, has never been reported.

So, there is still a need to develop a sustainable cyclisation process in the presence of a small amount of heterogeneous catalyst while improving the conversion and the selectivity.

The present invention allows to solve the above problem by using heterogenous acidic catalyst in order to prepare a cycloether of formula (I). To the best of our knowledge, the invention's conditions have never been reported in the prior art.

### SUMMARY OF THE INVENTION

The invention relates to a novel process allowing the preparation of a cycloether formula (I) by the cyclisation of a compound of formula (II) in a presence of a small amount of heterogeneous acidic catalyst never reported or suggested in the prior art.

So, a first object of the present invention is a process for the preparation of a cycloether of formula (I)

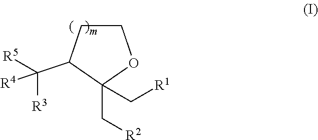

in the form of any one of its stereoisomers or a mixture thereof;
wherein m is 1 or 2;
R¹ represents a hydrogen atom or a C₁₋₃ alkyl group;
each R², R³, R⁴ represent, when taken separately, independently from each other, a hydrogen atom or a C₁₋₁₈ alkyl group optionally comprising one or two functional groups selected amongst ether, ester, carbonyl, amine, amide or alcohol group; or
R¹ and R², when taken together, represent a C₂₋₁₁ alkanediyl group optionally comprising one or two functional groups selected amongst ether, ester, carbonyl, amine, amide or alcohol group and/or R² and R³, when taken together, represent a C₁₋₁₁ alkanediyl group and/or R³ and R⁴, when taken together, represent a C₄₋₉ alkanediyl group and/or R¹ and R⁴, when taken together, represent a C₂₋₉ alkanediyl group;
R⁵ represents a hydrogen atom or a C₁₋₃ alkyl group;

comprising the cyclisation of compound of formula (II)

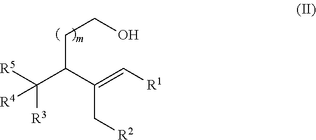

in the form of any one of its stereoisomers or a mixture thereof;
   wherein m, R¹, R², R³, R⁴ and R⁵ have the same meaning as defined above;
in the presence of heterogenous acidic catalyst.

A second object of the invention is a composition of matter comprising:
a) at least 95% of (3aR,5aS,9aS,9bR)-3a,6,6,9a-tetramethyldodecahydronaphtho[2,1-b]furan;
b) at most 5% of (3aS,5aS,9aS,9bR)-3a,6,6,9a-tetramethyldodecahydronaphtho[2,1-b]furan;
the percentage being relative to the total weight of the composition of matter.

### DESCRIPTION OF THE INVENTION

Surprisingly, it has now been discovered that the cyclisation of compound of formula (II) in a presence of a small amount of heterogenous acidic catalyst allows preparing a compound of formula (I) with high yield and high selectivity. The invention's process allows limiting even preventing the isomerization of the double bond in a more stable position; i.e. the isomerization of a double bond in an endo position; while maintaining even improving the formation of desired isomers; i.e. limiting the formation of the undesired diastereoisomer(s).

Therefore, a first object of the present invention is a process for the preparation of a cycloether of formula (I)

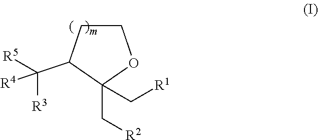

in the form of any one of its stereoisomers or a mixture thereof;
wherein m is 1 or 2;
R¹ represents a hydrogen atom or a C₁₋₃ alkyl group;
each R², R³, R⁴ represent, when taken separately, independently from each other, a hydrogen atom or a C₁₋₁₈ alkyl group optionally comprising one or two functional groups selected amongst ether, ester, carbonyl, amine, amide or alcohol group; or
R¹ and R², when taken together, represent a C₂₋₁₁ alkanediyl group optionally comprising one or two functional groups selected amongst ether, ester, carbonyl, amine, amide or alcohol group and/or R² and R³, when taken together, represent a C₁₋₁₁ alkanediyl group and/or R³ and R⁴, when taken together, represent a C₄₋₉ alkanediyl group and/or R¹ and R⁴, when taken together, represent a C₂₋₉ alkanediyl group;
R⁵ represents a hydrogen atom or a C₁₋₃ alkyl group;
comprising the cyclisation of compound of formula (II)

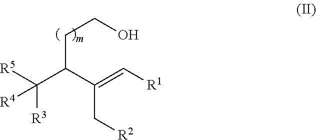

in the form of any one of its stereoisomers or a mixture thereof;
   wherein m, R¹, R², R³, R⁴ and R⁵ have the same meaning as defined;
in the presence of heterogenous acidic catalyst.

For the sake of clarity, by the expression “any one of its stereoisomers or a mixture thereof”, or the similar, it is meant the normal meaning understood by a person skilled in the art, i.e. that the compounds cited in the invention can be a pure enantiomer or a mixture of enantiomers. In other words, the compounds cited in the invention may possess at least one stereocenter which can have two different stereochemistries (e.g. R or S), e.g. the R² group may comprise at least one stereocenter. Said compounds may even be in the form of a pure enantiomer or in the form of a mixture of enantiomers. The compounds cited in the invention may even be in the form of a pure diastereoisomer or in the form of a mixture of diastereoisomers when said compounds possess more than one stereocenter. Said compounds can be in a racemic form or scalemic form. Therefore, said compounds can be one stereoisomer or in the form of a composition of matter comprising, or consisting of, various stereoisomers.

The term “optionally” is understood that a group can or cannot comprise a certain functional group.

The terms “alkyl” and “alkanediyl” are understood as comprising linear, branched, cyclic or alicyclic alkyl and alkanediyl groups.

It is understood that with the terms “ . . . optionally comprising one or two functional groups selected amongst ether, ester, carbonyl, amine, amide or alcohol group . . . ” it is meant that the groups can either substitute a hydrogen atom of the alkyl group and thus be laterally attached to said alkyl group, or substitute a carbon atom (if chemically possible) of the alkyl group and thus be inserted into the alkyl chain. For example, a —CH₂—CH₂—CHOH—CH₂— group represents a C₄ alkyl group comprising an alcohol group (substitution of a hydrogen atom), a —CH₂—CH₂—COO—CH₂—CH₂—OCO—CH₂—CH₂— group represents a C₆ alkyl group comprising two ester groups (substitution of carbon atoms/insertion into the alkyl chain) and, similarly, a —CH₂—CH₂—O—CH₂—CH₂—O—CH₂—CH₂— group represents a C₆ alkyl group comprising two ether groups.

It is understood that by “ . . . . R¹ and R², when taken together, represent a C₂₋₁₁ alkanediyl group . . . and/or R² and R³, when taken together, represent a C₁₋₁₁ alkanediyl group and/or R³ and R⁴, when taken together, represent . . . and/or R¹ and R⁴, when taken together . . . ” or the similar, that said group could form a (poly)cyclic alkyl group. In other words compound (II) could be acyclic, monocyclic, bicyclic or tricyclic and compound (I) could be monocyclic, bicyclic, tricyclic or teracyclic, e.g. in the case wherein R² and R³, as 20 well as R³ and R⁴, are taken together, the compound of formula (II) comprises a bicyclic moiety such as a decalin, e.g. R², R³ and R⁴, taken together, represents an alkanetriyl.

According to a particular embodiment, compound of formula (II) may be in a form of a composition of matter comprising compound of formula (II) and compound of formula (II′)

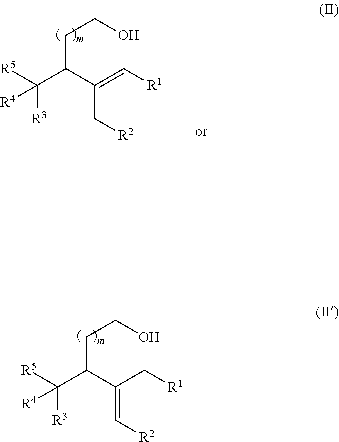

wherein m, R¹, R², R³, R⁴ and R⁵ have the same meaning as defined above. Particularly, compound of formula (II) may be in a form of a composition of matter comprising compound of formula (II) and compound of formula (II′). Particularly, compound of formula (II) may be in a form of a composition of matter comprising at least 50% of compound of formula (II) and at most 50% of compound of formula (II′), the percentage being relative to the total weight of the composition of matter. Particularly, compound of formula (II) may be in a form of a composition of matter comprising at least 60% of compound of formula (II) and at most 40% of compound of formula (II′). Particularly, compound of formula (II) may be in a form of a composition of matter comprising at least 70% of compound of formula (II) and at most 30% of compound of formula (II′). Particularly, compound of formula (II) may be in a form of a composition of matter comprising at least 80% of compound of formula (II) and at most 20% of compound of formula (II′). Particularly, compound of formula (II) may be in a form of a composition of matter comprising at least 90% of compound of formula (II) and at most 10% of compound of formula (II′). Particularly, compound of formula (II) may be in a form of a composition of matter comprising at least 95% of compound of formula (II) and at most 5% of compound of formula (II′). Particularly, compound of formula (II) may be in a form of a composition of matter comprising at least 98% of compound of formula (II) and at most 2% of compound of formula (II′). Particularly, compound of formula (II) may be in a form of a composition of matter comprising at least 99% of compound of formula (II) and at most 1% of compound of formula (II′). Particularly, compound of formula (II) may be in a form of a composition of matter comprising at least 99.5% of compound of formula (II) and at most 0.5% of compound of formula (II′). Even more particularly, the compound of formula (II) is free of compound of formula (II′).

According to any embodiment of the present invention, the heterogenous acidic catalyst may be amorphous or crystalline, particularly crystalline.

According to a particular embodiment of the invention, the heterogenous acidic catalyst may be an acidic resin. Non-limiting examples of suitable acidic resin may include A-15 dry, A-35 dry, A-36 dry sold by Dupont.

According to any embodiment of the present invention, the heterogenous acidic catalyst comprises silicon, tin, zirconium, hafnium or titanium and a second metal selected from the group consisting of aluminum, boron, iron or a mixture thereof. Particularly, the heterogenous acidic catalyst may be an aluminosilicate catalyst. Particularly, the aluminosilicate catalyst may be a zeolite or a clay.

According to any embodiment of the present invention, the commercial available clay may contain water. Said water may be removed partly or totally before use. The person skilled in the art is well aware of methods to remove water such as, for example, azeotropic distillation, vacuum striping or heating under nitrogen flow.

According to any embodiment of the present invention, the clay may be naturally occurring. Particularly, the clay is an acid-treated clay. Non-limiting examples of suitable clays may include F-20X, F20-XLM, F-21X, F-24X, F-22, F-118FF presently sold by EP Minerals, Fulcat-22F and Fulcat-22B sold by Byk, K-5, K-10-S300, K-20, K-21, K-30, K-41 sold by Clariant.

According to any embodiment of the present invention, the zeolite is a large pore zeolite.

By “the term large pore zeolite” it is meant the normal meaning in the art; i.e. 12 membered ring zeolite having pore size comprised in the range between 6.0 Angstrom and 7.5 Angstrom.

According to any embodiment of the present invention, the zeolite has a FAU, BEA and MOR topology. Particularly, the zeolite has a FAU topology.

According to any embodiment of the present invention, the Silicon: Aluminum ratio is comprised in the range between 2.5:1 and 300:1. Particularly, the Silicon: Aluminum ratio is comprised in the range between 5:1 and 150:1. Particularly, the Silicon: Aluminum ratio is comprised in the range between 10:1 and 150:1. Even more particularly, Particularly, the Silicon: Aluminum ratio is comprised in the range between 10:1 and 70:1.

According to any embodiment of the present invention, the zeolite is used in its protonic form. The latter can be directly provided by the manufacturer and used as such or obtained by calcination of the ammonium-exchange form if required. Particularly, the zeolite may be preactivated before reaction. The preactivation may be carry out by heating the zeolite at a temperature comprised between 300° C. and 600° C. for at least 1 hour.

Non-limiting examples of suitable zeolites may include CBV720, CBV760, CBV780 presently sold by Zeolyst Corporation.

The heterogenous acidic catalyst can be added into the reaction medium of the invention's process to form the cycloether of formula (I) in a large range of concentrations. As non-limiting examples, one can cite, as heterogenous acidic catalyst concentration values those ranging from 0.5 wt % to 20 wt %, relative to the total amount of the cycloether of formula (I). Particularly, the heterogenous acidic catalyst concentration may be comprised between 1 wt % to 15 wt %. Even more particularly, the heterogenous acidic catalyst concentration may be comprised between 3 wt % to 10 wt %. It goes without saying that the process works also with more catalyst. However the optimum concentration of heterogenous acidic catalyst will depend, as the person skilled in the art knows, on the nature of the latter, on the nature of the substrate, on the temperature and on the desired time of reaction.

The heterogenous acidic catalyst is commercially available compound or can be prepared by several methods, such as the one reported in U.S. Pat. No. 20,040,141,911, U.S. Pat. Nos. 6,054,113, 4,809,302,470,872 and EP0398636.

According to any one of the invention's embodiments, the invention's process for the preparation of a cycloether of formula (I) is carried out at a temperature comprised between 0° C. and 150° C. In particular, the temperature is in the range between 30° C. and 70° C. Of course, a person skilled in the art is also able to select the preferred temperature as a function of the melting and boiling point of the starting and final products as well as the desired time of reaction, conversion or selectivity.

The invention's process for the preparation of a cycloether of formula (I) can be carried out in the presence or absence of a solvent. When a solvent is required or used for practical reasons, then any solvent current in such reaction type can be used for the purposes of the invention. Non-limiting examples include C₆₋₁₂ aromatic solvents such as xylene, toluene, 1,3-diisopropylbenzene, cumene pseudocumene, anisole or chlorobenzene or mixtures thereof, hydrocarbon solvents such as cyclohexane, heptane or mixtures thereof, nitrile solvent such as acetonitrile, esteral solvents such as ethyl acetate or ethereal solvents such as tetrahydrofuran, diethyether, methyl tetrahydrofuran or mixtures thereof. The choice of the solvent is function of the nature of the substrate and/or catalyst and the person skilled in the art is well able to select the solvent most suitable in each case to optimize the reaction.

The invention's process for the preparation of a cycloether of formula (I) is carried out under batch or continuous conditions.

The invention's process for the preparation of a cycloether of formula (I) may be performed under atmospheric pressure or under a slight vacuum.

According to any one of the invention's embodiments, the cycloether of formula (I) is a polycyclic compound. The term “polycyclic compound” is understood that compound of formula (I) comprises at least 2 rings; e.g. the cycloether of formula (I) being a bicyclic compound. Particularly, the cycloether of formula (I) may be a bicyclic, tricyclic, tetracyclic or pentacyclic compound. Even more particularly, the cycloether of formula (I) may be a fused bicyclic, tricyclic or tetracyclic compound.

According to any one of the above embodiments of the invention, the cycloether of formula (I) is a C₉-C₂₀ compound.

According to any one of the above embodiments of the invention, R¹ may be a hydrogen atom or a C₁₋₃ linear or branched alkyl group. Particularly, R¹ may be a hydrogen atom, a methyl or a ethyl group. Particularly, R¹ may be a hydrogen atom or a methyl group. Even more particularly, R¹ may be a hydrogen atom.

According to any one of the invention's embodiments, m may be 1. In other word, compound of formula (I) is a tetrahydrofuran derivative.

According to any one of the invention's embodiments, R² and R³ may be taken together, and represent a C₁₋₁₁ alkanediyl group. Particularly, R² and R³ may be taken together, and represent a C₁₋₁₁ linear or branched alkanediyl group.

According to any one of the invention's embodiments, the cycloether is a compound of formula

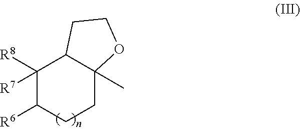

in the form of any one of its stereoisomers or a mixture thereof;
wherein n is 0 or 1;
each R⁶, R⁷ represent, when taken separately, independently from each other, a hydrogen atom or a C₁₋₉ alkyl group optionally comprising one or two functional groups selected amongst ether, ester, carbonyl, amine, amide or alcohol group; or
R⁶ and R⁷, when taken together, represent a C₃₋₁₀ linear or branched alkanediyl group; and
R⁸ represents a hydrogen atom or a C₁₋₃ linear or branched alkyl group.

According to any one of the invention's embodiments, the compound of formula (II) is a compound of formula

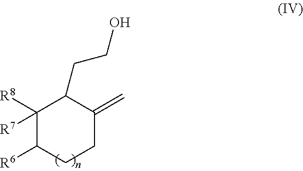

in the form of any one of its stereoisomers or a mixture thereof;
wherein n is 0 or 1;
each R⁶, R⁷ represent, when taken separately, independently from each other, a hydrogen atom or a C₁₋₉ alkyl group optionally comprising one or two functional groups selected amongst ether, ester, carbonyl, amine, amide or alcohol group; or
R⁶ and R⁷, when taken together, represent a C₃₋₁₀ linear or branched alkanediyl group; and
R⁸ represents a hydrogen atom or a C₁₋₃ linear or branched alkyl group.

According to a particular embodiment compounds (III) may be a tricyclic compound and the compound of formula (IV) may be a bicylic compound. Said compound of formula (IV) can be synthetic or natural.

According to any one of the invention's embodiments, R⁶ and R⁷ may represent, when taken separately, independently from each other, a hydrogen atom or a C₁₋₉ alkyl group optionally comprising one or two functional groups selected amongst ether, ester, carbonyl, amine, amide or alcohol group; or R⁶ and R⁷, when taken together, represent a C₃₋₉ linear or branched alkanediyl group. Particularly, R⁶ and R⁷ may represent, when taken separately, independently from each other, a hydrogen atom or a C₁₋₆ alkyl group optionally substituted comprising one or two functional groups selected amongst ether, ester, carbonyl, amine, amide or alcohol group; or R⁶ and R⁷, when taken together, may represent a C₃₋₈ linear or branched alkanediyl group. Particularly, R⁶ and R⁷, may represent, when taken separately, independently from each other, a hydrogen atom or a C₁₋₄ alkyl group optionally substituted comprising one or two functional groups selected amongst ether, ester, carbonyl, amine, amide or alcohol group; or R⁶ and R⁷, when taken together, may represent a C₃₋₈ linear or branched alkanediyl group.

According to any one of the invention's embodiments, said R⁶ group may represent a hydrogen atom or C₁₋₃ alkyl group. Particularly, R⁶ group may represent a hydrogen atom or a methyl group. Even more particularly, R⁶ group may represent a hydrogen atom.

According to any one of the invention's embodiments, said R⁷ group may represent a hydrogen atom or C₁₋₃ alkyl group. Particularly, R⁷ group may represent a hydrogen atom or a methyl group. Even more particularly, R⁷ group may represent a hydrogen atom.

According to any one of the invention's embodiments, said R⁶ and R⁷, when taken together, may represent a C₃₋₆ linear or branched alkanediyl group or even preferably a C₆ branched alkanediyl group.

According to any one of the invention's embodiments, n may be 1.

According to any one of the invention's embodiments, the cycloether may be a compound of formula

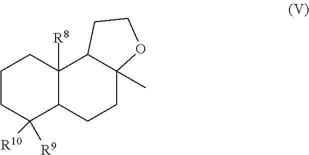

in the form of any one of its stereoisomers or a mixture thereof;
wherein R⁸ has the same meaning as defined above and each R⁹ and R¹⁰ represent, independently from each other, a C₁₋₃ linear or branched alkyl group.

According to any one of the invention's embodiments, the compound of formula (II) is a compound of formula

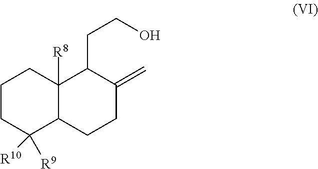

in the form of any one of its stereoisomers or a mixture thereof;
wherein R⁸ has the same meaning as defined above and each R⁹ and R¹⁰
represent, independently from each other, a C₁₋₃ linear or branched alkyl group. According to any one of the invention's embodiments, R⁸ may be a C₁₋₃ linear alkyl group. Particularly, R⁸ may be a methyl or ethyl group. Even more particularly, R⁸ may be a methyl group.

According to any one of the invention's embodiments, R⁹ may be methyl or ethyl group. Even more particularly, R⁹ may be a methyl group.

According to any one of the invention's embodiments, R¹⁰ may be methyl or ethyl group. Even more particularly, R¹⁰ may be a methyl group.

According to a particular embodiment of the invention, the compound of formula (II) may be 2-(5,5,8a-trimethyl-2-methylenedecahydronaphthalen-1-yl)ethan-1-ol and the corresponding the cycloether of formula (I) may be 3a,6,6,9a-tetramethyldodecahydronaphtho[2,1-b]furan which have four stereogenic centers being in a configuration R or S or a mixture thereof. In other worlds 2-(5,5,8a-trimethyl-2-methylenedecahydronaphthalen-1-yl)ethan-1-ol may be in the form of an essentially pure stereoisomer or in the form of a mixture of stereoisomers. According to a particular embodiment of the invention, the compound of formula (II), (IV) or (VI) may be 2-(5,5,8a-trimethyl-2-methylenedecahydronaphthalen-1-yl)ethan-1-ol in the form of a mixture of stereoisomers containing at least 50% of 2-((1SR,4aSR,8aSR)-5,5,8a-trimethyl-2-methylenedecahydronaphthalen-1-yl)ethan-1-ol. Even more particularly, the compound of formula (II), (IV) or (VI) may be 2-(5,5,8a-trimethyl-2-methylenedecahydronaphthalen-1-yl)ethan-1-ol in the form of a mixture of stereoisomers containing at least 75% of 2-((1SR,4aSR,8aSR)-5,5,8a-trimethyl-2-methylenedecahydronaphthalen-1-yl)ethan-1-ol. According to a particular embodiment of the invention, the compound of formula (II), (IV) or (VI) may be 2-((1SR,4aSR,8aSR)-5,5,8a-trimethyl-2-methylenedecahydronaphthalen-1-yl)ethan-1-ol. For the sake of clarity, by the expression “1SR,4aSR,8aRS” it is meant an equimolar mixture of 1S,4aS,8aR and 1R,4aR,8aS. According to particular embodiment of the invention, the compound of formula (II), (IV) or (VI) may be 2-((1S,4aS,8aS)-5,5,8a-trimethyl-2-methylenedecahydronaphthalen-1-yl)ethan-1-ol.

According to a particular embodiment of the invention, the compound of formula (II), (IV) or (VI) may be may be in a form of a composition of matter comprising at least 50% of 2-((1SR,4aSR,8aSR)-5,5,8a-trimethyl-2-methylenedecahydronaphthalen-1-yl)ethan-1-ol and at most 50% of 2-((1SR,4aSR,8aSR)-2,5,5,8a-tetramethyl-1,4,4a,5,6,7,8,8a-octahydronaphthalen-1-yl)ethan-1-ol. Particularly, the compound of formula (II), (IV) or (VI) may be may be in a form of a composition of matter comprising at least 60% of 2-((1SR,4aSR,8aSR)-5,5,8a-trimethyl-2-methylenedecahydronaphthalen-1-yl)ethan-1-ol and at most 40% of 2-((1SR,4aSR,8aSR)-2,5,5,8a-tetramethyl-1,4,4a,5,6,7,8,8a-octahydronaphthalen-1-yl)ethan-1-ol. Particularly, the compound of formula (II), (IV) or (VI) may be may be in a form of a composition of matter comprising at least 70% of 2-((1SR,4aSR,8aSR)-5,5,8a-trimethyl-2-methylenedecahydronaphthalen-1-yl)ethan-1-ol and at most 30% of 2-((1SR,4aSR,8aSR)-2,5,5,8a-tetramethyl-1,4,4a,5,6,7,8,8a-octahydronaphthalen-1-yl)ethan-1-ol. Particularly, the compound of formula (II), (IV) or (VI) may be may be in a form of a composition of matter comprising at least 80% of 2-((1SR,4aSR,8aSR)-5,5,8a-trimethyl-2-methylenedecahydronaphthalen-1-yl)ethan-1-ol and at most 20% of 2-((1SR,4aSR,8aSR)-2,5,5,8a-tetramethyl-1,4,4a,5,6,7,8,8a-octahydronaphthalen-1-yl)ethan-1-ol. Particularly, the compound of formula (II), (IV) or (VI) may be may be in a form of a composition of matter comprising at least 90% of 2-((1SR,4aSR,8aSR)-5,5,8a-trimethyl-2-methylenedecahydronaphthalen-1-yl)ethan-1-ol and at most 10% of 2-((1SR,4aSR,8aSR)-2,5,5,8a-tetramethyl-1,4,4a,5,6,7,8,8a-octahydronaphthalen-1-yl)ethan-1-ol. Particularly, the compound of formula (II), (IV) or (VI) may be may be in a form of a composition of matter comprising at least 95% of 2-((1SR,4aSR,8aSR)-5,5,8a-trimethyl-2-methylenedecahydronaphthalen-1-yl)ethan-1-ol and at most 5% of 2-((1SR,4aSR,8aSR)-2,5,5,8a-tetramethyl-1,4,4a,5,6,7,8,8a-octahydronaphthalen-1-yl)ethan-1-ol. Particularly, the compound of formula (II), (IV) or (VI) may be may be in a form of a composition of matter comprising at least 98% of 2-((1SR,4aSR,8aSR)-5,5,8a-trimethyl-2-methylenedecahydronaphthalen-1-yl)ethan-1-ol and at most 2% of 2-((1SR,4aSR,8aSR)-2,5,5,8a-tetramethyl-1,4,4a,5,6,7,8,8a-octahydronaphthalen-1-yl)ethan-1-ol. Particularly, the compound of formula (II), (IV) or (VI) may be may be in a form of a composition of matter comprising at least 99% of 2-((1SR,4aSR,8aSR)-5,5,8a-trimethyl-2-methylenedecahydronaphthalen-1-yl)ethan-1-ol and at most 1% of 2-((1SR,4aSR,8aSR)-2,5,5,8a-tetramethyl-1,4,4a,5,6,7,8,8a-octahydronaphthalen-1-yl)ethan-1-ol. Even more particularly, the compound of formula (II), (IV) or (VI) may be may be in a form of a composition of matter comprising at least 99.5% of 2-((1SR,4aSR,8aSR)-5,5,8a-trimethyl-2-methylenedecahydronaphthalen-1-yl)ethan-1-ol and at most 0.5% of 2-((1SR,4aSR,8aSR)-2,5,5,8a-tetramethyl-1,4,4a,5,6,7,8,8a-octahydronaphthalen-1-yl)ethan-1-ol.

According to a particular embodiment of the invention, the compound of formula (II), (IV) or (VI) may be may be in a form of a composition of matter comprising at least 50% of 2-((1S,4aS,8aS)-5,5,8a-trimethyl-2-methylenedecahydronaphthalen-1-yl)ethan-1-ol and at most 50% of 2-((1S,4aS,8aS)-2,5,5,8a-tetramethyl-1,4,4a,5,6,7,8,8a-octahydronaphthalen-1-yl)ethan-1-ol. Particularly, the compound of formula (II), (IV) or (VI) may be may be in a form of a composition of matter comprising at least 60% of 2-((1S,4aS,8aS)-5,5,8a-trimethyl-2-methylenedecahydronaphthalen-1-yl)ethan-1-ol and at most 40% of 2-((1S,4aS,8aS)-2,5,5,8a-tetramethyl-1,4,4a,5,6,7,8,8a-octahydronaphthalen-1-yl)ethan-1-ol. Particularly, the compound of formula (II), (IV) or (VI) may be may be in a form of a composition of matter comprising at least 70% of 2-((1S,4aS,8aS)-5,5,8a-trimethyl-2-methylenedecahydronaphthalen-1-yl)ethan-1-ol and at most 30% of 2-((1S,4aS,8aS)-2,5,5,8a-tetramethyl-1,4,4a,5,6,7,8,8a-octahydronaphthalen-1-yl)ethan-1-ol. Particularly, the compound of formula (II), (IV) or (VI) may be may be in a form of a composition of matter comprising at least 80% of 2-((1S,4aS,8aS)-5,5,8a-trimethyl-2-methylenedecahydronaphthalen-1-yl)ethan-1-ol and at most 20% of 2-((1S,4aS,8aS)-2,5,5,8a-tetramethyl-1,4,4a,5,6,7,8,8a-octahydronaphthalen-1-yl)ethan-1-ol. Particularly, the compound of formula (II), (IV) or (VI) may be may be in a form of a composition of matter comprising at least 90% of 2-((1S,4aS,8aS)-5,5,8a-trimethyl-2-methylenedecahydronaphthalen-1-yl)ethan-1-ol and at most 10% of 2-((1S,4aS,8aS)-2,5,5,8a-tetramethyl-1,4,4a,5,6,7,8,8a-octahydronaphthalen-1-yl)ethan-1-ol. Particularly, the compound of formula (II), (IV) or (VI) may be may be in a form of a composition of matter comprising at least 95% of 2-((1S,4aS,8aS)-5,5,8a-trimethyl-2-methylenedecahydronaphthalen-1-yl)ethan-1-ol and at most 5% of 2-((1S,4aS,8aS)-2,5,5,8a-tetramethyl-1,4,4a,5,6,7,8,8a-octahydronaphthalen-1-yl)ethan-1-ol. Particularly, the compound of formula (II), (IV) or (VI) may be may be in a form of a composition of matter comprising at 98% of 2-((1S,4aS,8aS)-5,5,8a-trimethyl-2-least methylenedecahydronaphthalen-1-yl)ethan-1-ol and at most 2% of 2-((1S,4aS,8aS)-2,5,5,8a-tetramethyl-1,4,4a,5,6,7,8,8a-octahydronaphthalen-1-yl)ethan-1-ol. Particularly, the compound of formula (II), (IV) or (VI) may be may be in a form of a composition of matter comprising at of 2-((1S,4aS,8aS)-5,5,8a-trimethyl-2-least 99% methylenedecahydronaphthalen-1-yl)ethan-1-ol and at most 1% of 2-((1S,4aS,8aS)-2,5,5,8a-tetramethyl-1,4,4a,5,6,7,8,8a-octahydronaphthalen-1-yl)ethan-1-ol. Even more particularly, the compound of formula (II), (IV) or (VI) may be may be in a form of a composition of matter comprising at least 99.5% of 2-((1S,4aS,8aS)-5,5,8a-trimethyl-2-methylenedecahydronaphthalen-1-yl)ethan-1-ol and at most 0.5% of 2-((1S,4aS,8aS)-2,5,5,8a-tetramethyl-1,4,4a,5,6,7,8,8a-octahydronaphthalen-1-yl)ethan-1-ol.

According to any embodiment of the invention's, the invention's process is stereoselective. In other words, the cyclodehydration of 2-((1S,4aS,8aS)-5,5,8a-trimethyl-2-methylenedecahydronaphthalen-1-yl)ethan-1-ol provides (3aR,5aS,9aS,9bR)-3a,6,6,9a-tetramethyldodecahydronaphtho[2,1-b]furan.

According to a particular embodiment of the invention, the compound of formula (I) may be 3a,6,6,9a-tetramethyldodecahydronaphtho[2,1-b]furan in the form of a mixture of stereoisomers containing at least 50% of (3aRS,5aSR,9aSR,9bRS)-3a,6,6,9a-tetramethyldodecahydronaphtho[2,1-b]furan. Even more particularly, the compound of formula (I) may be 3a,6,6,9a-tetramethyldodecahydronaphtho[2,1-b]furan in the form of a mixture of stereoisomers containing at least 75% of (3aRS,5aSR,9aSR,9bRS)-3a,6,6,9a-tetramethyldodecahydronaphtho[2,1-b]furan. According to a particular embodiment of the invention, the compound of formula (I) may be (3aRS,5aSR,9aSR,9bRS)-3a,6,6,9a-tetramethyldodecahydronaphtho[2,1-b]furan. For the sake of clarity, by the expression “3aRS,5aSR,9aSR,9bRS” it is meant an equimolar mixture of 3aR,5aS,9aS,9bR and 3aS,5aR,9aR,9bS. According to particular embodiment of the invention, the compound of formula (I) may be (3aR,5aS,9aS,9bR)-3a,6,6,9a-tetramethyldodecahydronaphtho[2,1-b]furan.

According to a particular embodiment of the invention, the compound of formula (I) may be may be in a form of a composition of matter comprising at least 95% of (3aRS,5aSR,9aSR,9bRS)-3a,6,6,9a-tetramethyldodecahydronaphtho[2,1-b]furan and at most 5% of (3aSR,5aSR,9aSR,9bRS)-3a,6,6,9a-tetramethyldodecahydronaphtho[2,1-b]furan. Particularly, the compound of formula (I) may be may be in a form of a composition of matter comprising at least 96% of (3aRS,5aSR,9aSR,9bRS)-3a,6,6,9a-tetramethyldodecahydronaphtho[2,1-b]furan and at most 4% of (3aSR,5aSR,9aSR,9bRS)-3a,6,6,9a-tetramethyldodecahydronaphtho[2,1-b]furan. Particularly, the compound of formula (I) may be may be in a form of a composition of matter comprising at least 97% of (3aRS,5aSR,9aSR,9bRS)-3a,6,6,9a-tetramethyldodecahydronaphtho[2,1-b]furan and at most 3% of (3aSR,5aSR,9aSR,9bRS)-3a,6,6,9a-tetramethyldodecahydronaphtho[2,1-b]furan. Particularly, the compound of formula (I) may be may be in a form of a composition of matter comprising at least 98% of (3aRS,5aSR,9aSR,9bRS)-3a,6,6,9a-tetramethyldodecahydronaphtho[2,1-b]furan and at most 2% of (3aSR,5aSR,9aSR,9bRS)-3a,6,6,9a-tetramethyldodecahydronaphtho[2,1-b]furan. Particularly, the compound of formula (I) may be may be in a form of a composition of matter comprising at least 99% of (3aRS,5aSR,9aSR,9bRS)-3a,6,6,9a-tetramethyldodecahydronaphtho[2,1-b]furan and at most 1% of (3aSR,5aSR,9aSR,9bRS)-3a,6,6,9a-tetramethyldodecahydronaphtho[2,1-b]furan. Particularly, the compound of formula (I) may be may be in a form of a composition of matter comprising at least 99.3% of (3aRS,5aSR,9aSR,9bRS)-3a,6,6,9a-tetramethyldodecahydronaphtho[2,1-b]furan and at most 0.7% of (3aSR,5aSR,9aSR,9bRS)-3a,6,6,9a-tetramethyldodecahydronaphtho[2,1-b]furan. Particularly, the compound of formula (I) may be may be in a form of a composition of matter comprising at least 99.4% of (3aRS,5aSR,9aSR,9bRS)-3a,6,6,9a-tetramethyldodecahydronaphtho[2,1-b]furan and at most 0.6% of (3aSR,5aSR,9aSR,9bRS)-3a,6,6,9a-tetramethyldodecahydronaphtho[2,1-b]furan. Even more particularly, the compound of formula (I) may be may be in a form of a composition of matter comprising at least 99.5% of (3aRS,5aSR,9aSR,9bRS)-3a,6,6,9a-tetramethyldodecahydronaphtho[2,1-b]furan and at most 0.5% of (3aSR,5aSR,9aSR,9bRS)-3a,6,6,9a-tetramethyldodecahydronaphtho[2,1-b]furan.

According to a particular embodiment of the invention, the compound of formula (I) may be may be in a form of a composition of matter comprising at least 95% of (3aR,5aS,9aS,9bR)-3a,6,6,9a-tetramethyldodecahydronaphtho[2,1-b]furan and at most 5% of (3aS,5aS,9aS,9bR)-3a,6,6,9a-tetramethyldodecahydronaphtho[2,1-b]furan. Particularly, the compound of formula (I) may be may be in a form of a composition of matter comprising at least 96% of (3aR,5aS,9aS,9bR)-3a,6,6,9a-tetramethyldodecahydronaphtho[2,1-b]furan and at most 4% of (3aS,5aS,9aS,9bR)-3a,6,6,9a-tetramethyldodecahydronaphtho[2,1-b]furan. Particularly, the compound of formula (I) may be may be in a form of a composition of matter comprising at least 97% of (3aR,5aS,9aS,9bR)-3a,6,6,9a-tetramethyldodecahydronaphtho[2,1-b]furan and at most 3% of (3aS,5aS,9aS,9bR)-3a,6,6,9a-tetramethyldodecahydronaphtho[2,1-b]furan. Particularly, the compound of formula (I) may be may be in a form of a composition of matter comprising at 98% least of (3aR,5aS,9aS,9bR)-3a,6,6,9a-tetramethyldodecahydronaphtho[2,1-b]furan and at most 2% of (3aS,5aS,9aS,9bR)-3a,6,6,9a-tetramethyldodecahydronaphtho[2,1-b]furan. Particularly, the compound of formula (I) may be may be in a form of a composition of matter comprising at least 99% of (3aR,5aS,9aS,9bR)-3a,6,6,9a-tetramethyldodecahydronaphtho[2,1-b]furan and at most 1% of (3aS,5aS,9aS,9bR)-3a,6,6,9a-tetramethyldodecahydronaphtho[2,1-b]furan. Particularly, the compound of formula (I) may be may be in a form of a composition of matter comprising at least 99.3% of (3aR,5aS,9aS,9bR)-3a,6,6,9a-tetramethyldodecahydronaphtho[2,1-b]furan and at most 0.7% of (3aS,5aS,9aS,9bR)-3a,6,6,9a-tetramethyldodecahydronaphtho[2,1-b]furan. Particularly, the compound of formula (I) may be may be in a form of a composition of matter comprising at least 99.4% of (3aR,5aS,9aS,9bR)-3a,6,6,9a-tetramethyldodecahydronaphtho[2,1-b]furan and at most 0.6% of (3aS,5aS,9aS,9bR)-3a,6,6,9a-tetramethyldodecahydronaphtho[2,1-b]furan. Even more particularly, the compound of formula (I) may be may be in a form of a composition of matter comprising at least 99.5% of (3aR,5aS,9aS,9bR)-3a,6,6,9a-tetramethyldodecahydronaphtho[2,1-b]furan and at most 0.5% of (3aS,5aS,9aS,9bR)-3a,6,6,9a-tetramethyldodecahydronaphtho[2,1-b]furan.

According to a particular embodiment of the invention, the compound of formula (I) may be may be in a form of a composition of matter comprising from 95% to 99.99% of (3aR,5aS,9aS,9bR)-3a,6,6,9a-tetramethyldodecahydronaphtho[2,1-b]furan and from 0.01% to 5% of (3aS,5aS,9aS,9bR)-3a,6,6,9a-tetramethyldodecahydronaphtho[2,1-b]furan. Particularly, the compound of formula (I) may be may be in a form of a composition of matter comprising from 96% to 99.99% of (3aR,5aS,9aS,9bR)-3a,6,6,9a-tetramethyldodecahydronaphtho[2,1-b]furan and from 0.01% to 4% of (3aS,5aS,9aS,9bR)-3a,6,6,9a-tetramethyldodecahydronaphtho[2,1-b]furan. Particularly, the compound of formula (I) may be may be in a form of a composition of matter comprising from 97% to 99.99% of (3aR,5aS,9aS,9bR)-3a,6,6,9a-tetramethyldodecahydronaphtho[2,1-b]furan and from 0.01% to 3% of (3aS,5aS,9aS,9bR)-3a,6,6,9a-tetramethyldodecahydronaphtho[2,1-b]furan. Particularly, the compound of formula (I) may be may be in a form of a composition of matter comprising from 98% to 99.99% of (3aR,5aS,9aS,9bR)-3a,6,6,9a-tetramethyldodecahydronaphtho[2,1-b]furan and from 0.01% to 2% of (3aS,5aS,9aS,9bR)-3a,6,6,9a-tetramethyldodecahydronaphtho[2,1-b]furan. Particularly, the compound of formula (I) may be may be in a form of a composition of matter comprising from 99% to 99.99% of (3aR,5aS,9aS,9bR)-3a,6,6,9a-tetramethyldodecahydronaphtho[2,1-b]furan and from 0.01% to 1% of (3aS,5aS,9aS,9bR)-3a,6,6,9a-tetramethyldodecahydronaphtho[2,1-b]furan. Particularly, the compound of formula (I) may be may be in a form of a composition of matter comprising from 99.3% to 99.99% of (3aR,5aS,9aS,9bR)-3a,6,6,9a-tetramethyldodecahydronaphtho[2,1-b]furan and from 0.01% to 0.7% of (3aS,5aS,9aS,9bR)-3a,6,6,9a-tetramethyldodecahydronaphtho[2,1-b]furan. Particularly, the compound of formula (I) may be may be in a form of a composition of matter comprising from 99.3% to 99.9% of (3aR,5aS,9aS,9bR)-3a,6,6,9a-tetramethyldodecahydronaphtho[2,1-b]furan and from 0.1% to 0.7% of (3aS,5aS,9aS,9bR)-3a,6,6,9a-tetramethyldodecahydronaphtho[2,1-b]furan. Particularly, the compound of formula (I) may be may be in a form of a composition of matter comprising from 99.4% to 99.9% of (3aR,5aS,9aS,9bR)-3a,6,6,9a-tetramethyldodecahydronaphtho[2,1-b]furan and from 0.1% to 0.6% of (3aS,5aS,9aS,9bR)-3a,6,6,9a-tetramethyldodecahydronaphtho[2,1-b]furan. Particularly, the compound of formula (I) may be may be in a form of a composition of matter comprising from 99.5% to 99.9% of (3aR,5aS,9aS,9bR)-3a,6,6,9a-tetramethyldodecahydronaphtho[2,1-b]furan and from 0.1% to 0.5% of (3aS,5aS,9aS,9bR)-3a,6,6,9a-tetramethyldodecahydronaphtho[2,1-b]furan. Particularly, the compound of formula (I) may be may be in a form of a composition of matter comprising from 99.5% to 99.8% of (3aR,5aS,9aS,9bR)-3a,6,6,9a-tetramethyldodecahydronaphtho[2,1-b]furan and from 0.2% to 0.5% of (3aS,5aS,9aS,9bR)-3a,6,6,9a-tetramethyldodecahydronaphtho[2,1-b]furan.

Unless stated otherwise, percentages (%) are meant to designate a percentage by weight of a composition.

The compound of formula (II), (IV) (VI) can be prepared by several methods known in the art, for example in the case of 2-(5,5,8a-trimethyl-2-methylenedecahydronaphthalen-1-yl)ethan-1-ol, said compound may be obtained as reported in Australian Journal of Chemistry, 1989, 497. The compound of formula (II), (IV) or (VI) may also be produced in vitro using purified recombinantly prepared enzymes or by fermentation using host cells, such as microbial cells, genetically engineered to convert unexpensive carbon sources (such as sugar) into the desired compound of formula (II), (IV) or (VI) or, in particular, into 2-(5,5,8a-trimethyl-2-methylenedecahydronaphthalen-1-yl)ethyl acetate in a form of any one of its stereoisomers or a mixture thereof. 2-(5,5,8a-trimethyl-2-methylenedecahydronaphthalen-1-yl)ethyl acetate may be converted into 2-(5,5,8a-trimethyl-2-methylenedecahydronaphthalen-1-yl)ethan-1-ol using chemical or enzymatic conditions known in the art. The advantage of using a compound of formula (II), (IV) or (VI) obtained by fermentation is evident since it allows an easy access to the starting material with high enantiomeric excess.

According to any one of the above embodiments of the invention's process, said process is further characterized in that the compound of formula (II), (IV) or (VI), in particular 2-((1S,4aS,8aS)-5,5,8a-trimethyl-2-methylenedecahydronaphthalen-1-yl)ethan-1-ol, is prepared, by a process comprising the step of contacting farnesyl pyrophosphate with at least one enzyme.

Another objet of the invention is a composition of matter comprising at least 95% of (3aRS,5aSR,9aSR,9bRS)-3a,6,6,9a-tetramethyldodecahydronaphtho[2,1-b]furan and at most 5% of (3aSR,5aSR,9aSR,9bRS)-3a,6,6,9a-tetramethyldodecahydronaphtho[2,1-b]furan. Particularly, the invention's composition of matter may comprise at least 96% of (3aRS,5aSR,9aSR,9bRS)-3a,6,6,9a-tetramethyldodecahydronaphtho[2,1-b]furan and at most 4% of (3aSR,5aSR,9aSR,9bRS)-3a,6,6,9a-tetramethyldodecahydronaphtho[2,1-b]furan. Particularly, the invention's composition of matter may comprise at least 97% of (3aRS,5aSR,9aSR,9bRS)-3a,6,6,9a-tetramethyldodecahydronaphtho[2,1-b]furan and at most 3% of (3aSR,5aSR,9aSR,9bRS)-3a,6,6,9a-tetramethyldodecahydronaphtho[2,1-b]furan. Particularly, the invention's composition of matter may comprise at least 98% of (3aRS,5aSR,9aSR,9bRS)-3a,6,6,9a-tetramethyldodecahydronaphtho[2,1-b]furan and at most 2% of (3aSR,5aSR,9aSR,9bRS)-3a,6,6,9a-tetramethyldodecahydronaphtho[2,1-b]furan. Particularly, the invention's composition of matter may comprise at least 99% of (3aRS,5aSR,9aSR,9bRS)-3a,6,6,9a-tetramethyldodecahydronaphtho[2,1-b]furan and at most 1% of (3aSR,5aSR,9aSR,9bRS)-3a,6,6,9a-tetramethyldodecahydronaphtho[2,1-b]furan. Particularly, the invention's composition of matter may comprise at least 99.3% of (3aRS,5aSR,9aSR,9bRS)-3a,6,6,9a-tetramethyldodecahydronaphtho[2,1-b]furan and at most 0.7% of (3aSR,5aSR,9aSR,9bRS)-3a,6,6,9a-tetramethyldodecahydronaphtho[2,1-b]furan. Particularly, the invention's composition of matter may comprise at least 99.4% of (3aRS,5aSR,9aSR,9bRS)-3a,6,6,9a-tetramethyldodecahydronaphtho[2,1-b]furan and at most 0.6% of (3aSR,5aSR,9aSR,9bRS)-3a,6,6,9a-tetramethyldodecahydronaphtho[2,1-b]furan. Even more particularly, the invention's composition of matter may comprise at least 99.5% of (3aRS,5aSR,9aSR,9bRS)-3a,6,6,9a-tetramethyldodecahydronaphtho[2,1-b]furan and 0.5% at most of (3aSR,5aSR,9aSR,9bRS)-3a,6,6,9a-tetramethyldodecahydronaphtho[2,1-b]furan.

According to a particular embodiment of the invention, the invention's composition of matter may comprise at least 95% of (3aR,5aS,9aS,9bR)-3a,6,6,9a-tetramethyldodecahydronaphtho[2,1-b]furan and at most 5% of (3aS,5aS,9aS,9bR)-3a,6,6,9a-tetramethyldodecahydronaphtho[2,1-b]furan. Particularly, the invention's composition of matter may comprise at least 96% of (3aR,5aS,9aS,9bR)-3a,6,6,9a-tetramethyldodecahydronaphtho[2,1-b]furan and at most 4% of (3aS,5aS,9aS,9bR)-3a,6,6,9a-tetramethyldodecahydronaphtho[2,1-b]furan. Particularly, the invention's composition of matter may comprise at least 97% of (3aR,5aS,9aS,9bR)-3a,6,6,9a-tetramethyldodecahydronaphtho[2,1-b]furan and at most 3% of (3aS,5aS,9aS,9bR)-3a,6,6,9a-tetramethyldodecahydronaphtho[2,1-b]furan. Particularly, the invention's composition of matter may comprise at least 98% of (3aR,5aS,9aS,9bR)-3a,6,6,9a-tetramethyldodecahydronaphtho[2,1-b]furan and at most 2% of (3aS,5aS,9aS,9bR)-3a,6,6,9a-tetramethyldodecahydronaphtho[2,1-b]furan. Particularly, the invention's composition of matter may comprise at least 99% of (3aR,5aS,9aS,9bR)-3a,6,6,9a-tetramethyldodecahydronaphtho[2,1-b]furan and at most 1% of (3aS,5aS,9aS,9bR)-3a,6,6,9a-tetramethyldodecahydronaphtho[2,1-b]furan. Particularly, the invention's composition of matter may comprise at least 99.3% of (3aR,5aS,9aS,9bR)-3a,6,6,9a-tetramethyldodecahydronaphtho[2,1-b]furan and at most 0.7% of (3aS,5aS,9aS,9bR)-3a,6,6,9a-tetramethyldodecahydronaphtho[2,1-b]furan. Particularly, the invention's composition of matter may comprise at least 99.4% of (3aR,5aS,9aS,9bR)-3a,6,6,9a-tetramethyldodecahydronaphtho[2,1-b]furan and at most 0.6% of (3aS,5aS,9aS,9bR)-3a,6,6,9a-tetramethyldodecahydronaphtho[2,1-b]furan. Even more particularly, the invention's composition of matter may comprise at least 99.5% of (3aR,5aS,9aS,9bR)-3a,6,6,9a-tetramethyldodecahydronaphtho[2,1-b]furan and at most 0.5% of (3aS,5aS,9aS,9bR)-3a,6,6,9a-tetramethyldodecahydronaphtho[2,1-b]furan.

According to a particular embodiment of the invention, the invention's composition of matter may comprise from 95% to 99.99% of (3aR,5aS,9aS,9bR)-3a,6,6,9a-tetramethyldodecahydronaphtho[2,1-b]furan and from 0.01% to 5% of (3aS,5aS,9aS,9bR)-3a,6,6,9a-tetramethyldodecahydronaphtho[2,1-b]furan. Particularly, the invention's composition of matter may comprise from 96% to 99.99% of (3aR,5aS,9aS,9bR)-3a,6,6,9a-tetramethyldodecahydronaphtho[2,1-b]furan and from 0.01% to 4% of (3aS,5aS,9aS,9bR)-3a,6,6,9a-tetramethyldodecahydronaphtho[2,1-b]furan. Particularly, the invention's composition of matter may comprise from 97% to 99.99% of (3aR,5aS,9aS,9bR)-3a,6,6,9a-tetramethyldodecahydronaphtho[2,1-b]furan and from 0.01% to 3% of (3aS,5aS,9aS,9bR)-3a,6,6,9a-tetramethyldodecahydronaphtho[2,1-b]furan. Particularly, the invention's composition of matter may comprise from 98% to 99.99% of (3aR,5aS,9aS,9bR)-3a,6,6,9a-tetramethyldodecahydronaphtho[2,1-b]furan and from 0.01% to 2% of (3aS,5aS,9aS,9bR)-3a,6,6,9a-tetramethyldodecahydronaphtho[2,1-b]furan. Particularly, the invention's composition of matter may comprise from 99% to 99.99% of (3aR,5aS,9aS,9bR)-3a,6,6,9a-tetramethyldodecahydronaphtho[2,1-b]furan and from 0.01% to 1% of (3aS,5aS,9aS,9bR)-3a,6,6,9a-tetramethyldodecahydronaphtho[2,1-b]furan. Particularly, the invention's composition of matter may comprise from 99.3% to 99.99% of (3aR,5aS,9aS,9bR)-3a,6,6,9a-tetramethyldodecahydronaphtho[2,1-b]furan and from 0.01% to 0.7% of (3aS,5aS,9aS,9bR)-3a,6,6,9a-tetramethyldodecahydronaphtho[2,1-b]furan. Particularly, the invention's composition of matter may comprise from 99.3% to 99.9% of (3aR,5aS,9aS,9bR)-3a,6,6,9a-tetramethyldodecahydronaphtho[2,1-b]furan and from 0.1% to 0.7% of (3aS,5aS,9aS,9bR)-3a,6,6,9a-tetramethyldodecahydronaphtho[2,1-b]furan. Particularly, the invention's composition of matter may comprise from 99.4% to 99.9% of (3aR,5aS,9aS,9bR)-3a,6,6,9a-tetramethyldodecahydronaphtho[2,1-b]furan and from 0.1% to 0.6% of (3aS,5aS,9aS,9bR)-3a,6,6,9a-tetramethyldodecahydronaphtho[2,1-b]furan. Particularly, the invention's composition of matter may comprise from 99.5% to 99.9% of (3aR,5aS,9aS,9bR)-3a,6,6,9a-tetramethyldodecahydronaphtho[2,1-b]furan and from 0.1% to 0.5% of (3aS,5aS,9aS,9bR)-3a,6,6,9a-tetramethyldodecahydronaphtho[2,1-b]furan. Particularly, the invention's composition of matter may comprise from 99.5% to 99.8% of (3aR,5aS,9aS,9bR)-3a,6,6,9a-tetramethyldodecahydronaphtho[2,1-b]furan and from 0.2% to 0.5% of (3aS,5aS,9aS,9bR)-3a,6,6,9a-tetramethyldodecahydronaphtho[2,1-b]furan.

Said invention's composition of matter can be used as perfuming ingredient, for instance to impart odor notes of the amber type. So another object of the invention is the use as perfuming ingredient of the invention's composition of matter as defined above. In other words, it concerns a method or a process to confer, enhance, improve or modify the odor properties of a perfuming composition or of a perfumed article or of a surface, which method comprises adding to said composition or article an effective amount of the invention's composition of matter, e.g. to impart its typical note. Understood that the final hedonic effect may depend on the precise dosage and on the organoleptic properties of the invention's composition of matter, but anyway the addition of the invention's composition of matter will impart to the final product its typical touch in the form of a note, touch or aspect depending on the dosage.

By “use of the the invention's composition of matter” it has to be understood here also the use of any composition containing the invention's composition of matter and which can be advantageously employed in the perfumery industry.

Said compositions, which in fact can be advantageously employed as perfuming ingredients, are also an object of the present invention.

Therefore, another object of the present invention is a perfuming composition comprising:
i) as a perfuming ingredient, at least the invention's composition of matter as defined above;
ii) at least one ingredient selected from the group consisting of a perfumery carrier and a perfumery base; and
iii) optionally at least one perfumery adjuvant.

By “perfumery carrier” it is meant here a material which is practically neutral from a perfumery point of view, i.e. that does not significantly alter the organoleptic properties of perfuming ingredients. Said carrier may be a liquid or a solid.

As liquid carrier one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting examples, solvents such as butylene or propylene glycol, glycerol, dipropyleneglycol and its monoether, 1,2,3-propanetriyl triacetate, dimethyl glutarate, dimethyl adipate 1,3-diacetyloxypropan-2-yl acetate, diethyl phthalate, isopropyl myristate, Abalyn® (rosin resins, available from Eastman), benzyl benzoate, benzyl alcohol, 2-(2-ethoxyethoxy)-1-ethanol, tri-ethyl citrate or mixtures thereof, which are the most commonly used or also naturally derived solvents like glycerol or various vegetable oils such as palm oil, sunflower oil or linseed oil. For the compositions which comprise both a perfumery carrier and a perfumery base, other suitable perfumery carriers than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company), or hydrogenated castors oils such as those known under the trademark Cremophor® RH 40 (origin: BASF).

Solid carrier is meant to designate a material to which the perfuming composition or some element of the perfuming composition can be chemically or physically bound. In general such solid carriers are employed either to stabilize the composition, or to control the rate of evaporation of the compositions or of some ingredients. Solid carriers are of current use in the art and a person skilled in the art knows how to reach the desired effect. However by way of non-limiting examples of solid carriers, one may cite absorbing gums or polymers or inorganic materials, such as porous polymers, cyclodextrines, dextrines, maltodextrines wood based materials, organic or inorganic gels, clays, gypsum talc or zeolites.

As other non-limiting examples of solid carriers, one may cite encapsulating materials. Examples of such materials may comprise wall-forming and plasticizing materials, such as glucose syrups, natural or modified starches, hydrocolloids, cellulose derivatives, polyvinyl acetates, polyvinylalcohols, proteins or pectins, plant gums such as acacia gum (Gum Arabic), urea, sodium chloride, sodium sulphate, zeolite, sodium carbonate, sodium bicarbonate, clay, talc, calcium carbonate, magnesium sulfate, gypsum, calcium sulfate, magnesium oxide, zinc oxide, titanium dioxide, calcium chloride, potassium chloride, magnesium chloride, zinc chloride, carbohydrates, saccharides such as sucrose, mono-, di-, tri- and polysaccharides and derivatives such as chitosan, starch, cellulose, carboxymethyl methylcellulose, methylcellulose, hydroxyethyl cellulose, ethyl cellulose, propyl cellulose, polyols/sugar alcohols such as sorbitol, maltitol, xylitol, erythritol, and isomalt, polyethylene glycol (PEG), polyvinyl pyrrolidin (PVP), polyvinyl alcohol, acrylamides, acrylates, polyacrylic acid and related, maleic anhydride copolymers, amine-functional polymers, vinyl ethers, styrenes, polystyrenesulfonates, vinyl acids, ethylene glycol-propylene glycol block copolymers, vegetable gums, gum acacia, pectins, xanthanes, alginates, carragenans, citric acid or any water soluble solid acid, fatty alcohols or fatty acids and mixtures thereof, or yet the materials cited in reference texts such as H. Scherz, Hydrokolloide: Stabilisatoren, Dickungs-und Geliermittel in Lebensmitteln, Band 2 der Schriftenreihe Lebensmittelchemie, Lebensmittelqualität, Behr's Verlag GmbH & Co., Hamburg, 1996. The encapsulation is a well-known process to a person skilled in the art, and may be performed, for instance, by using techniques such as spray-drying, agglomeration or yet extrusion; or consists of a coating encapsulation, including coacervation and complex coacervation techniques.

As non-limiting examples of solid carriers, one may cite in particular the core-shell capsules with resins of aminoplast, polyamide, polyester, polyurea or polyurethane type or a mixture thereof (all of said resins are well known to a person skilled in the art) using techniques like phase separation process induced by polymerization, interfacial polymerization, coacervation or altogether (all of said techniques have been described in the prior art), optionally in the presence of a polymeric stabilizer or of a cationic copolymer.

Resins may be produced by the polycondensation of an aldehyde (e.g. formaldehyde, 2,2-dimethoxyethanal, glyoxal, glyoxylic acid or glycolaldehyde and mixtures thereof) with an amine such as urea, benzoguanamine, glycouryl, melamine, methylol melamine, methylated methylol melamine, guanazole and the like, as well as mixtures thereof. Alternatively one may use preformed resins alkylolated polyamines such as those commercially available under the trademark Urac® (origin: Cytec Technology Corp.), Cymel® (origin: Cytec Technology Corp.), Urecoll® or Luracoll® (origin: BASF).

Other resins are the ones produced by the polycondensation of a polyol, like glycerol, and a polyisocyanate, like a trimer of hexamethylene diisocyanate, a trimer of isophorone diisocyanate or xylylene diisocyanate or a Biuret of hexamethylene diisocyanate or a trimer of xylylene diisocyanate with trimethylolpropane (known with the tradename of Takenate®, origin: Mitsui Chemicals), among which a trimer of xylylene diisocyanate with trimethylolpropane and a Biuret of hexamethylene diisocyanate are preferred.

Some of the seminal literature related to the encapsulation of perfumes by polycondensation of amino resins, namely melamine based resins with aldehydes includes articles such as those published by K. Dietrich et al. Acta Polymerica, 1989, vol. 40, pages 243, 325 and 683, as well as 1990, vol. 41, page 91. Such articles already describe the various parameters affecting the preparation of such core-shell microcapsules following prior art methods that are also further detailed and exemplified in the patent literature. US 4′396′670, to the Wiggins Teape Group Limited is a pertinent early example of the latter. Since then, many other authors have enriched the literature in this field and it would be impossible to cover all published developments here, but the general knowledge in encapsulation technology is very significant. More recent publications of pertinence, which disclose suitable uses of such microcapsules, are represented for example by the article of K. Bruyninckx and M. Dusselier, ACS Sustainable Chemistry & Engineering, 2019, vol. 7, pages 8041-8054.

By “perfumery base” what is meant here is a composition comprising at least one perfuming co-ingredient.

Said perfuming co-ingredient is not (3aR,5aS,9aS,9bR)-3a,6,6,9a-tetramethyldodecahydronaphtho[2,1-b]furan. Moreover, by “perfuming co-ingredient” it is meant here a compound, which is used in a perfuming preparation or a composition to impart a hedonic effect; i.e. used for the primary purpose of conferring or modulating an odor. In other words such a co-ingredient, to be considered as being a perfuming one, must be recognized by a person skilled in the art as being able to impart or modify in a positive or pleasant way the odor of a composition, and not just as having an odor. The perfuming co-ingredient may impart an additional benefit beyond that of modifying or imparting an odor, such as long-lasting, blooming, malodour counteraction, antimicrobial effect, antiviral effect, microbial stability, or pest control.

The nature and type of the perfuming co-ingredients present in the base do not warrant a more detailed description here, which in any case would not be exhaustive, the skilled person being able to select them on the basis of his general knowledge and according to the intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulphurous heterocyclic compounds and essential oils, and said perfuming co-ingredients can be of natural or synthetic origin.

In particular one may cite perfuming co-ingredients which are commonly used in perfume formulations, such as:
Aldehydic ingredients: decanal, dodecanal, 2-methyl-undecanal, 10-undecenal, octanal, nonanal and/or nonenal;
Aromatic-herbal ingredients: eucalyptus oil, camphor, eucalyptol, 5-methyltricyclo[6.2.1.0˜2,7˜]undecan-4-one, 1-methoxy-3-hexanethiol, 2-ethyl-4,4-dimethyl-1,3-oxathiane, 2,2,7/8,9/10-Tetramethylspiro[5.5]undec-8-en-1-one, menthol and/or alpha-pinene;
Balsamic ingredients: coumarin, ethylvanillin and/or vanillin;
Citrus ingredients: dihydromyrcenol, citral, orange oil, linalyl acetate, citronellyl nitrile, orange terpenes, limonene, 1-p-menthen-8-yl acetate and/or 1,4(8)-p-menthadiene;
Floral ingredients:methyl dihydrojasmonate, linalool, citronellol, phenylethanol, 3-(4-tert-butylphenyl)-2-methylpropanal, hexylcinnamic aldehyde, benzyl acetate, benzyl salicylate, tetrahydro-2-isobutyl-4-methyl-4(2H)-pyranol, beta ionone, methyl 2-(methylamino)benzoate, (E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, 1-(2,6,6-trimethyl-1,3-cyclohexadien-1-yl)-2-buten-1-one, (2E)-1-(2,6,6-trimethyl-2-cyclohexen-1-yl)-2-buten-1-one, (2E)-1-[2,6,6-trimethyl-3-cyclohexen-1-yl]-2-buten-1-one, (2E)-1-(2,6,6-trimethyl-1-cyclohexen-1-yl)-2-buten-1-one, 2,5-dimethyl-2-indanmethanol, 2,6,6-trimethyl-3-cyclohexene-1-carboxylate, 3-(4,4-3-(3,3/1,1-dimethyl-5-indanyl)propanal, dimethyl-1-cyclohexen-1-yl)propanal, hexyl salicylate, 3,7-dimethyl-1,6-nonadien-3-ol, 3-(4-isopropylphenyl)-2-methylpropanal, verdyl acetate, geraniol, p-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, verdyl proprionate, geranyl acetate, tetrahydro linalool, cis-7-p-menthanol, propyl (S)-2-(1,1-dimethylpropoxy)propanoate, 2-methoxynaphthalene, 2,2,2-trichloro-1-phenylethyl acetate, 4/3-(4-hydroxy-4-methylpentyl)-3-cyclohexene-1-carbaldehyde, amylcinnamic aldehyde, 8-decen-5-olide, 4-phenyl-2-butanone, isononyle acetate, 4-(1,1-dimethylethyl)-1-cyclohexyl acetate, verdyl isobutyrate and/or mixture of methylionones isomers;
Fruity ingredients: gamma-undecalactone, 2,2,5-trimethyl-5-pentylcyclopentanone, 2-methyl-4-propyl-1,3-oxathiane, 4-decanolide, ethyl 2-methyl-pentanoate, hexyl acetate, ethyl 2-methylbutanoate, gamma-nonalactone, allyl heptanoate, 2-phenoxyethyl isobutyrate, ethyl 2-methyl-1,3-dioxolane-2-acetate, diethyl 1,4-cyclohexanedicarboxylate, 3-methyl-2-hexen-1-yl acetate, 1-[3,3-dimethylcyclohexyl]ethyl [3-ethyl-2-oxiranyl]acetate and/or diethyl 1,4-cyclohexane dicarboxylate;
Green ingredients: 2-methyl-3-hexanone (E)-oxime, 2,4-dimethyl-3-cyclohexene-1-carbaldehyde, 2-tert-butyl-1-cyclohexyl acetate, styrallyl acetate, allyl (2-methylbutoxy)acetate, 4-methyl-3-decen-5-ol, diphenyl ether, (Z)-3-hexen-1-ol and/or 1-(5,5-dimethyl-1-cyclohexen-1-yl)-4-penten-1-one;
Musk ingredients: 1,4-dioxa-5,17-cycloheptadecanedione, (Z)-4-cyclopentadecen-1-one, 3-methylcyclopentadecanone, 1-oxa-12-cyclohexadecen-2-one, 1-oxa-13-cyclohexadecen-2-one, (9Z)-9-cycloheptadecen-1-one, 2-{(1S)-1-[(1R)-3,3-dimethylcyclohexyl]ethoxy}-2-oxoethyl propionate, 3-methyl-5-cyclopentadecen-1-one, 4,6,6,7,8,8-hexamethyl-1,3,4,6,7,8-hexahydrocyclopenta[g]isochromene, (1S,1′R)-2-[1-(3′,3′-dimethyl-1′-cyclohexyl)ethoxy]-2-methylpropyl propanoate, oxacyclohexadecan-2-oneand/or (1S,1′R)-[1-(3′,3′-dimethyl-1′-cyclohexyl)ethoxycarbonyl]methyl propanoate;
Woody ingredients: 1-[(1RS,6SR)-2,2,6-trimethylcyclohexyl]-3-hexanol, 3,3-dimethyl-5-[(1R)-2,2,3-trimethyl-3-cyclopenten-1-yl]-4-penten-2-ol, 3,4′-dimethylspiro[oxirane-2,9′-tricyclo[6.2.1.0^{2.7}]undec[4]ene, (1-ethoxyethoxy)cyclododecane, 2,2,9,11-tetramethylspiro[5.5]undec-8-en-1-yl acetate, 1-(octahydro-2,3,8,8-tetramethyl-2-naphtalenyl)-1-ethanone, patchouli oil, terpenes fractions of patchouli oil, Clearwood®® (Origin: Firmenich SA), (1′R,E)-2-ethyl-4-(2′,2′,3′-trimethyl-3′-cyclopenten-1′-yl)-2-buten-1-ol, 2-ethyl-4-(2,2,3-trimethyl-3-cyclopenten-1-yl)-2-buten-1-ol, methyl cedryl ketone, 5-(2,2,3-trimethyl-3-cyclopentenyl)-3-methylpentan-2-ol, 1-(2,3,8,8-tetramethyl-1,2,3,4,6,7,8,8a-octahydronaphthalen-2-yl)ethan-1-one and/or isobornyl acetate;
Other ingredients (e.g. amber, powdery spicy or watery): heliotropin, anisic aldehyde, eugenol, cinnamic aldehyde, clove oil, 3-(1,3-benzodioxol-5-yl)-2-methylpropanal, 7-methyl-2H-1,5-benzodioxepin-3(4H)-one, 2,5,5-trimethyl-1,2,3,4,4a,5,6,7-octahydro-2-naphthalenol, 1-phenylvinyl acetate, 6-methyl-7-oxa-1-thia-4-azaspiro[4.4]nonane and/or 3-(3-isopropyl-1-phenyl)butanal.

A perfumery base according to the invention may not be limited to the above mentioned perfuming co-ingredients, and many other of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that said co-ingredients may also be compounds known to release in a controlled manner various types of perfuming compounds also known as properfume or profragrance. Non-limiting examples of suitable properfume may include 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, trans-3-(dodecylthio)-1-(2,6,6-trimethyl-3-cyclohexen-1-yl)-1-butanone, 3-(dodecylsulfonyl)-1-(2,6,6-trimethylcyclohex-3-en-1-yl)butan-1-one, a linear polysiloxane co-polymer of (3-mercaptopropyl)(methyl)dimethoxysilane, 1-[6-ethyl-2,6-dimethyl-3-cyclohexen-1-yl]-2-buten-1-one, 2-(dodecylthio)octan-4-one, 2-(dodecylsulfonyl)octan-4-one, 4-oxooctan-2-yl dodecanoate, 2-phenylethyl oxo(phenyl)acetate, 3,7-dimethylocta-2,6-dien-1-yl oxo(phenyl)acetate, (Z)-hex-3-en-1-yl oxo(phenyl)acetate, 3,7-dimethyl-2,6-octadien-1-yl hexadecanoate, bis(3,7-dimethylocta-2,6-dien-1-yl) succinate, (2E,6Z)-2,6-nonadienyl hexadecanoate, (2E,6Z)-2,6-nonadien-1-yl tetradecanoate, (2E,6Z)-2,6-nonadien-1-yl dodecanoate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene, (2-((2-pentylcyclopentylidene)methoxy)ethyl)benzene, 4-allyl-2-methoxy-1-((2-methoxy-2-phenylvinyl)oxy)benzene, (2-((2-heptylcyclopentylidene)methoxy)ethyl)benzene, 1-methoxy-4-(1-phenethoxyprop-1-en-2-yl)benzene, (2-((2-methyl-4-(2,6,6-trimethylcyclohex-1-en-1-yl)but-1-en-1-yl)oxy)ethyl)benzene, 1-methoxy-4-(2-methyl-3-phenethoxyallyl)benzene, methylcyclohexylidene)methoxy)ethyl)benzene, (2-((2-isopropyl-5-1-isopropyl-4-methyl-2-((2-pentylcyclopentylidene)methoxy)benzene, 2-methoxy-1-((2-pentylcyclopentylidene)methoxy)-4-propylbenzene, 2-ethoxy-1-((2-methoxy-2-phenylvinyl)oxy)-4-methylbenzene, 3-methoxy-4-((2-methoxy-2-phenylvinyl)oxy)benzaldehyde, 1-isopropyl-2-((2-methoxy-2-phenylvinyl)oxy)-4-methylbenzene, 4-((2-(hexyloxy)-2-phenylvinyl)oxy)-3-methoxybenzaldehyde or a mixture thereof.

By “perfumery adjuvant”, it is meant here an ingredient capable of imparting additional added benefit such as a color, a particular light resistance, chemical stability, etc. A detailed description of the nature and type of adjuvant commonly used in perfuming composition cannot be exhaustive, but it has to be mentioned that said ingredients are well known to a person skilled in the art. One may cite as specific non-limiting examples the following: viscosity agents (e.g. surfactants, thickeners, gelling and/or rheology modifiers), stabilizing agents (e.g. preservatives, antioxidant, heat/light and or buffers or chelating agents, such as BHT), coloring agents (e.g. dyes and/or pigments), preservatives (e.g. antibacterial or antimicrobial or antifungal or anti irritant agents), abrasives, skin cooling agents, fixatives, insect repellants, ointments, vitamins and mixtures thereof. By “fixative” also called “modulator”, it is understood here an agent having the capacity to affect the manner in which the odour, and in particular the evaporation rate and intensity, of the compositions incorporating said modulator can be perceived by an observer or user thereof, over time, as compared to the same perception in the absence of the modulator. In particular, the modulator allows prolonging the time during which their fragrance is perceived. Non-limiting examples of suitable modulators may include methyl glucoside polyol; ethyl glucoside polyol; propyl glucoside polyol; isocetyl alcohol; PPG-3 myristyl ether; neopentyl glycol diethylhexanoate; sucrose laurate; sucrose dilaurate, sucrose myristate, sucrose palmitate, sucrose stearate, sucrose distearate, sucrose tristearate, hyaluronic acid disaccharide sodium salt, sodium hyaluronate, propylene glycol propyl ether; dicetyl ether; polyglycerin-4 ethers; isoceteth-5; isoceteth-7, isoceteth-10; isoceteth-12; isoceteth-15; isoceteth-20; isoceteth-25; isoceteth-30; disodium lauroamphodipropionate; hexaethylene glycol monododecyl ether; and their mixtures; neopentyl glycol diisononanoate; cetearyl ethylhexanoate; panthenol ethyl ether, DL-panthenol, N-hexadecyl n-nonanoate, noctadecyl n-nonanoate, a profragrance, cyclodextrin, an encapsulation, and a combination thereof. At most 20% by weight, based on the total weight of the perfuming composition, of the modulator may be incorporated into the perfumed consumer product.

It is understood that a person skilled in the art is perfectly able to design optimal formulations for the desired effect by admixing the above mentioned components of a perfuming composition, simply by applying the standard knowledge of the art as well as by trial and error methodologies.

An invention's composition consisting of the invention's composition of matter and at least one perfumery carrier consists of a particular embodiment of the invention as well as a perfuming composition comprising the invention's composition of matter, at least one perfumery carrier, at least one perfumery base, and optionally at least one perfumery adjuvant.

For the sake of clarity, it is also understood that any mixture resulting directly from a chemical synthesis, e.g. a reaction medium without an adequate purification, in which the invention's composition of matter would be involved as a starting, intermediate or end-product could not be considered as a perfuming composition according to the invention as far as said mixture does not provide the inventive composition of matter in a suitable form for perfumery. Thus, unpurified reaction mixtures are generally excluded from the present invention unless otherwise specified.

The invention's composition of matter can also be advantageously used in all the fields of modern perfumery, i.e. fine or functional perfumery, to positively impart or modify the odor of a consumer product into which said invention's composition of matter is added. Consequently, another object of the present invention consists of a perfumed consumer product comprising, as a perfuming ingredient, the invention's composition of matter, as defined above.

The invention's composition of matter can be added as such or as part of an invention's perfuming composition.

For the sake of clarity, “perfumed consumer product” is meant to designate a consumer product which delivers at least a pleasant perfuming effect to the surface or space to which it is applied (e.g. skin, hair, textile, or home surface). In other words, a perfumed consumer product according to the invention is a perfumed consumer product which comprises a functional formulation, as well as optionally additional benefit agents, corresponding to the desired consumer product, and an olfactive effective amount of at least one invention's compound. For the sake of clarity, said perfumed consumer product is a non-edible product.

The nature and type of the constituents of the perfumed consumer product do not warrant a more detailed description here, which in any case would not be exhaustive, the skilled person being able to select them on the basis of his general knowledge and according to the nature and the desired effect of said product.

Non-limiting examples of suitable perfumed consumer products include a perfume, such as a fine perfume, a splash or eau de parfum, a cologne or a shave or after-shave lotion; a fabric care product, such as a liquid or solid detergent optionally in the form of a pod or tablet, a fabric softener, a liquid or solid scent booster, a dryer sheet, a fabric refresher, an ironing water, a paper, a bleach, a carpet cleaner, a curtain-care product; a body-care product, such as a hair care product (e.g. a shampoo, a leave-on or rinse-off hair conditioner, a coloring preparation or a hair spray, a color-care product, a hair shaping product, a dental care product), a disinfectant, an intimate care product; a cosmetic preparation (e.g. a skin cream or lotion, a vanishing cream or a deodorant or antiperspirant (e.g. a spray or roll on), a hair remover, a tanning or sun or after sun product, a nail product, a skin cleansing, a makeup); or a skin-care product (e.g. a soap, a shower or bath mousse, oil or gel, or a hygiene product or a foot/hand care products); an air care product, such as an air freshener or a “ready to use” powdered air freshener which can be used in the home space (rooms, refrigerators, cupboards, shoes or car) and/or in a public space (halls, hotels, malls, etc. . . . ); or a home care product, such as a mold remover, a furnisher care product, a wipe, a dish detergent or a hard-surface (e.g. a floor, bath, sanitary or a window-cleaning) detergent; a leather care product; a car care product, such as a polish, a wax or a plastic cleaner.

Some of the above-mentioned perfumed consumer products may represent an aggressive medium for the invention's composition of matter, so that it may be necessary to protect the latter from premature decomposition, for example by encapsulation or by chemically binding it to another chemical which is suitable to release the invention's composition of matter upon a suitable external stimulus, such as an enzyme, light, heat or a change of pH.

Typical manners to execute the invention's process are reported herein below in the examples.

### EXAMPLES

The invention will now be described in further detail by way of the following examples, wherein the abbreviations have the usual meaning in the art, the temperatures are indicated in degrees Centigrade (° C.). The preparation of. Gas chromatography was performed on an Agilent 7890 A Series equipped with a HP5 column (30 m×0.25 mm ID, 0.25 μm film) and tetradecane was used as internal standard.

### Example 1

### Typical Experimental Procedure for the Preactivation of the Zeolite Catalyst:

In the case of zeolite, a preactivation before testing was performed by placing 10 g of the as-received material in a muffle furnace (static air) heated at 10° C. min⁻¹ to 550° C. for 3 h. As this step is mandatory to convert samples in ammonium form to their protonic form, preactivation by calcination was systematically applied for every zeolite for sake of comparison.

### Example 2

### Preparation of (3aR,5aS,9aS,9bR)-3a,6,6,9a-tetramethyldodecahydronaphtho[2,1-b]furan by cyclisation of 2-((1S,4aS,8aS)-5,5,8a-trimethyl-2-methylenedecahydronaphthalen-1-yl)ethan-1-ol

2-((1S,4aS,8aS)-5,5,8a-trimethyl-2-methylenedecahydronaphthalen-1-yl)ethan-1-ol (Compound of formula (II), 5 g, 21.16 mmol) and toluene (95 g, 1.03 mol) were charged in a 100 mL round bottom flask and heated to 40° C. under mechanical stirring. Once the desired reaction temperature was reached, 250 mg (5 wt. % vs. starting material) of a solid-acid catalyst comprising either an acid-treated clay, an acidic-zeolite or an acidic resin was added and the mixture was left under stirring for 22 h maximum. Table I reported the yield of the desired (3aR,5aS,9aS,9bR)-3a,6,6,9a-tetramethyldodecahydronaphtho[2,1-b]furan (Compound of formula (I)), its isomer (3aS,5aS,9aS,9bR)-3a,6,6,9a-tetramethyldodecahydronaphtho[2,1-b]furan (corresponding to Compound A) and the starting material isomers 2-[(1S,4aS,8aS)-2,5,5,8a-tetramethyl-1,4,4a,5,6,7,8,8a-octahydro-1-naphthalenyl]ethanol and 2-((4aS,8aS)-2,5,5,8a-tetramethyl-3,4,4a,5,6,7,8,8a-octahydronaphthalen-1-yl)ethan-1-ol (corresponding respectively to Compound of Formula (II′) and Compound B).

TABLE 1
Preparation of (3aR,5aS,9aS,9bR)-3a,6,6,9a-tetramethyldodecahydronaphtho[2,1-b]furan by
cyclisation of 2-((1S,4aS,8aS)-5,5,8a-trimethyl-2-methylenedecahydronaphthalen-1-yl)ethan-1-ol

| | | | Compound | Compound | Compound | | |
|---|---|---|---|---|---|---|---|
| | | | of formula | of formula | of formula | Compound | Compound |
| | | Time | (I) | (II) | (II′) | B | A |
| Entry | Catalyst | (h) | (GC %) | (GC %) | (GC %) | (GC %) | (GC %) |
| H+ Clays | | | | | | | |
| 1 | F-20X | 22 | 90.53 | 8.73 | 0.46 | 0 | 0 |
| 2 | F-22 | 22 | 94.38 | 3.79 | 0.44 | 1.1 | 0 |
| 3 | F-24X | 7.5 | 95.83 | 2.14 | 0.39 | 1.34 | 0 |
| | | 22 | 97.01 | 0.79 | 0.31 | 1.55 | 0 |
| 4 | F-118FF | 22 | 80.63 | 18.7 | 0.43 | 0 | 0 |
| 5 | 116-FF | 7.5 | 80.12 | 18.36 | 0.46 | 0 | 0 |
| 6 | F-9 | 7.5 | 71.35 | 27.87 | 0.47 | 0 | 0 |
| 7 | EXBC-001 | 7.5 | 68.59 | 30.87 | 0.36 | 0 | 0 |
| 8 | EPZE | 7.5 | 47.63 | 52.06 | 0.31 | 0 | 0 |
| Zeolites | | | | | | | |
| 9 | CBV712 | 22 | 47.84 | 51.71 | 0.16 | 0 | 0.23 |
| 10 | CBV760 | 22 | 96.86 | 1.65 | 0 | 1.25 | 0.43 |
| 11 | CBV780 | 7.5 | 97.23 | 2.5 | 0 | 0 | 0.34 |
| | | 22 | 98.52 | 0.45 | 0 | 0.83 | 0.39 |
| 12 | CBV901 | 22 | 65.68 | 33.65 | 0.22 | 0 | 0.28 |
| SAC Resins | | | | | | | |
| 13 | A-15 Dry | 22 | 66.47 | 20.43 | 9.34 | 0 | 3.58 |
| 14 | A-35 Dry | 22 | 72.05 | 16.97 | 6.47 | 0 | 4.32 |
| Comparative | | | | | | | |
| examples | | | | | | | |
| 15 | FeCl₃^{a)} | 1 | 37.95 | 0.63 | 12.37 | 6.27 | 1.35 |
| 16 | Methane- | 0.5 | 54.32 | 1.38 | 20.12 | 5.79 | 13.0 |
| | sulfonic | | | | | | |
| | acid^{b)} | | | | | | |
| 17 | CBV780^{c)} | 22 | 0 | 0 | 0 | 100 | 0 |
| 18 | CBV780^{d)} | 22 | 1 | 0 | 93 | 6 | 0 |

^{a)}As described in WO 2009/053884 at room temperature using Compound II (5 wt. % in toluene) as a substrate and FeCl₃ (20 wt. % vs substrate) in catalytic amount
^{b)}As described in WO 2006/010287 at room temperature using Compound II (5 wt. % in toluene) as a substrate and methanesulfonic acid (3 mL) as catalyst
^{c)}Starting material is pure compound B
^{d)}Starting material is pure compound II'

The process of the present invention allows obtaining the compound of formula (I) very selectively with low or no formation of undesired compounds such as (3aS,5aS,9aS,9bR)-3a,6,6,9a-tetramethyldodecahydronaphtho[2,1-b]furan (Compound A), 2-[(1S,4aS,8aS)-2,5,5,8a-tetramethyl-1,4,4a,5,6,7,8,8a-octahydro-1-naphthalenyl]ethanol (Compound of Formula (II′)) and 2-((4aS,8aS)-2,5,5,8a-tetramethyl-3,4,4a,5,6,7,8,8a-octahydronaphthalen-1-yl)ethan-1-ol (Compound B). The cyclisation of compound of formula (II) in the presence of homogeneous catalyst as reported in the prior arts (Table 1, entry 15 and entry 16) provides a higher amount of the isomerized products; i.e. compound of formula (II′) and compound B while the cyclisation of compound B (Table 1, entry 17) or compound of formula (II′) (Table 1, entry 19) in the presence of heterogeneous catalyst does not provide compound of formula (I) or only traces of compound of formula (I).

### Example 3

### Preparation of (3aR,5aS,9aS,9bR)-3a,6,6,9a-tetramethyldodecahydronaphtho[2,1-b]furan by cyclisation of 2-((1S,4aS,8aS)-5,5,8a-trimethyl-2-methylenedecahydronaphthalen-1-yl)ethan-1-ol using different solvents

Experiment 2 was repeated using CBV780 but using different solvents as summarized in Table 2.

TABLE 2
Preparation of (3aR,5aS,9aS,9bR)-3a,6,6,9a-tetramethyldodecahydronaphtho[2,1-
b]furan by cyclisation of 2-((1S,4aS,8aS)-5,5,8a-trimethyl-2-methylenedecahydronaphthalen-
1-yl)ethan-1-ol using different solvents

| | | Compound | Compound | Compound | | |
|---|---|---|---|---|---|---|
| | | of formula | of formula | of formula | Compound | Compound |
| | | (I) | (II) | (II′) | B | A |
| Entry | Solvent | (GC %) | (GC %) | (GC %) | (GC %) | (GC %) |
| 1 | Cyclohexane | 76.63 | 16.86 | 6.03 | 0 | 0 |
| 2 | Toluene | 93.98 | 5.62 | 0.08 | 0 | 0 |
| 3 | Dichloromethane | 95.07 | 3.42 | 0 | 0 | 0 |

### Example 4

### Preparation of (3aR,5aS,9aS,9bR)-3a,6,6,9a-tetramethyldodecahydronaphtho[2,1-b]furan by cyclisation of 2-((1S,4aS,8aS)-5,5,8a-trimethyl-2-methylenedecahydronaphthalen-1-yl)ethan-1-ol

50 g of 2-((1S,4aS,8aS)-5,5,8a-trimethyl-2-methylenedecahydronaphthalen-1-yl)ethan-1-ol, 450 g of toluene were charged in a reactor and heated up to 40° C. Once the desired temperature was reached, 2.5 g of CBV780 was added and the reaction left under stirring for 12 h yielding 97.7 GC % of the desired (3aR,5aS,9aS,9bR)-3a,6,6,9a-tetramethyldodecahydronaphtho[2,1-b]furan product.

### Example 5

### Preparation of (3aR,5aS,9aS,9bR)-3a,6,6,9a-tetramethyldodecahydronaphtho[2,1-b]furan by cyclisation of 22-((1S,4aS,8aS)-5,5,8a-trimethyl-2-methylenedecahydronaphthalen-1-yl)ethan-1-ol

50 g of 2-((1S,4aS,8aS)-5,5,8a-trimethyl-2-methylenedecahydronaphthalen-1-yl)ethan-1-ol, 115 g of toluene were charged in a reactor and heated up to 50° C. Once the desired temperature was reached, 2.5 g of CBV780 was added and the reaction left under stirring for 12 h yielding 94.3 GC % of the desired (3aR,5aS,9aS,9bR)-3a,6,6,9a-tetramethyldodecahydronaphtho[2,1-b]furan product.

### Example 6

### Preparation of (3aR,5aS,9aS,9bR)-3a,6,6,9a-tetramethyldodecahydronaphtho[2,1-b]furan by cyclisation of 2-((1S,4aS,8aS)-5,5,8a-trimethyl-2-methylenedecahydronaphthalen-1-yl)ethan-1-ol

50 g of 2-((1S,4aS,8aS)-5,5,8a-trimethyl-2-methylenedecahydronaphthalen-1-yl)ethan-1-ol, 200 g of toluene were charged in a reactor and heated up to 50° C. Once the desired temperature was reached, 2.5 g of F-24X was added and the reaction left under stirring for 12 h yielding 88 GC % of the desired (3aR,5aS,9aS,9bR)-3a,6,6,9a-tetramethyldodecahydronaphtho[2,1-b]furan product.

The invention claimed is:

1. A process for the preparation of a cycloether of formula (I) 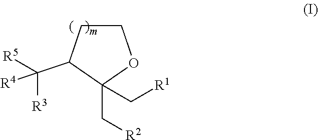
in the form of any one of its stereoisomers or a mixture thereof;
wherein m is 1 or 2;
R¹ represents a hydrogen atom or a C₁₋₃ alkyl group;
each R², R³, R⁴ represent, when taken separately, independently from each other, a hydrogen atom or a C₁₋₁₈ alkyl group optionally comprising one or two functional groups selected amongst ether, ester, carbonyl, amine, amide or alcohol group; or R¹ and R², when taken together, represent a C₂₋₁₁ alkanediyl group optionally comprising one or two functional groups selected amongst ether, ester, carbonyl, amine, amide or alcohol group and/or R² and R³, when taken together, represent a C₁₋₁₁ alkanediyl group and/or R³ and R⁴, when taken together, represent a C₄₋₉ alkanediyl group and/or R¹ and R⁴, when taken together, represent a C₂₋₉ alkanediyl group;
R⁵ represents a hydrogen atom or a C₁₋₃ alkyl group;
comprising the cyclisation of compound of formula (II) 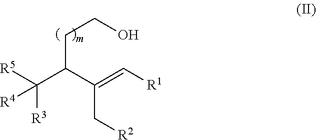
in the form of any one of its stereoisomers or a mixture thereof;
wherein m, R¹, R², R³, R⁴ and R⁵ have the same meaning as defined above;
in the presence of heterogenous acidic catalyst.

2. The process according to claim 1, wherein the heterogenous acidic catalyst is crystalline or amorphous.

3. The process according to claim 1, wherein the heterogenous acidic catalyst comprises silicon, tin, zirconium, hafnium or titanium and a second metal selected from the group consisting of aluminum, boron, iron or a mixture thereof.

4. The process according to claim 1, wherein the heterogenous acidic catalyst is an aluminosilicate catalyst.

5. The process according to claim 1, wherein the aluminosilicate catalyst is a zeolite or a clay.

6. The process according to claim 1, wherein the clay is an acid-treated clay.

7. The process according to claim 1, wherein the zeolite is a large pore zeolite.

8. The process according to claim 7, wherein the zeolite has a FAU, BEA or MOR topology.

9. The process according to claim 7, wherein the Silicon: Aluminum ratio is comprised in the range between 2.5; and 300:1.

10. The process according to claim 1, wherein R¹ is a hydrogen atom and m is 1.

11. The process according to claim 1, wherein the cycloether is a compound of formula 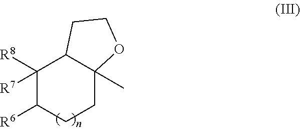
in the form of any one of its stereoisomers or a mixture thereof;
wherein n is 0 or 1;
each R⁶ and R⁷ represent, when taken separately, independently from each other, a hydrogen atom or a C₁₋₉ alkyl group optionally comprising one or two functional groups selected amongst ether, ester, carbonyl, amine, amide or alcohol group; or
R⁶ and R⁷, when taken together, represent a C₃₋₁₀ linear or branched alkanediyl group; and
R⁸ represents a hydrogen atom or a C₁₋₃ linear or branched alkyl group.

12. The process according to claim 1, wherein the compound of formula (II) is a compound of formula 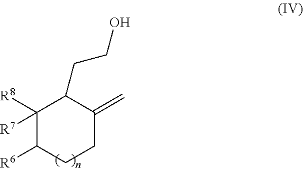
in the form of any one of its stereoisomers or a mixture thereof;
wherein n is 0 or 1;
each R⁶ and R⁷ represent, when taken separately, independently from each other, a hydrogen atom or a C₁₋₉ alkyl group optionally comprising one or two functional groups selected amongst ether, ester, carbonyl, amine, amide or alcohol group; or
R⁶ and R⁷, when taken together, represent a C₃₋₁₀ linear or branched alkanediyl group; and
R⁸ represents a hydrogen atom or a C₁₋₃ linear or branched alkyl group.

13. The process according to claim 1, wherein the cycloether is a compound of formula 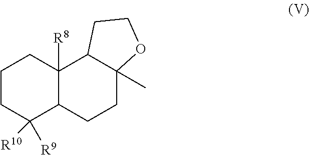
in the form of any one of its stereoisomers or a mixture thereof;
wherein R⁸ represents a hydrogen atom or a C₁₋₃ linear or branched alkyl group and each R⁹ and R¹⁰ represent, independently from each other, a C₁₋₃ linear or branched alkyl group.

14. The process according to claim 1, wherein the compound of formula (IV) is a compound of formula 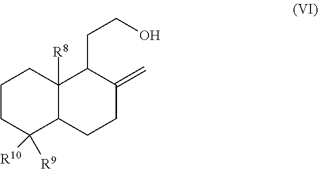
in the form of any one of its stereoisomers or a mixture thereof;
wherein R⁸ represents a hydrogen atom or a C₁₋₃ linear or branched alkyl group and each R⁹ and R¹⁰ represent, independently from each other, a C₁₋₃ linear or branched alkyl group.

15. The process according to claim 1, wherein the compound of formula (II) is prepared by a process comprising the step of contacting farnesyl pyrophosphate with at least one enzyme.

16. A composition of matter comprising:
a) at least 95% of (3aR,5aS,9aS,9bR)-3a,6,6,9a-tetramethyldodecahydronaphtho[2,1-b]furan;
b) at most 5% of (3aS,5aS,9aS,9bR)-3a,6,6,9a-tetramethyldodecahydronaphtho[2,1-b]furan;
the percentage being relative to the total weight of the composition of matter.
